(19) United States
(12) Patent Application Publication
(10) Pub. No.: US 2021/0290205 A1
(43) Pub. Date: Sep. 23, 2021

(54) Analytical Toilet for Detecting Viruses in Feces

(71) Applicant: Medic, Inc., Provo, UT (US)

(72) Inventors: David R. Hall, Provo, UT (US); K. Jeffrey Campbell, Spanish Fork, UT (US); Anthony E. Pullen, Tucson, AZ (US); Jeffery Duncan, Tucson, AZ (US)

(21) Appl. No.: 17/210,474

(22) Filed: Mar. 23, 2021

Related U.S. Application Data
(60) Provisional application No. 62/993,648, filed on Mar. 23, 2020.

(51) Int. Cl. A61B 10/00; C12Q 1/686; C12Q 1/70; B01L 3/00; E03D 9/00; G01N 33/483

(52) U.S. Cl. CPC A61B 10/0038; C12Q 1/686; C12Q 1/701; G01N 21/6428; E03D 9/00; G01N 33/4833; B01L 3/502715

(57) ABSTRACT

An analytical toilet is disclosed. The analytical toilet includes a bowl adapted to receive excreta and a detection system to detect a virus, such as a coronavirus. The analytical toilet presents a sample extracted from feces to the detection system and the detection system creates a signal indicating whether or not a coronavirus is present in the sample. Preferably, the detection system comprises a quantitative PCR instrument and a fluorimeter.

### CROSS-REFERENCE TO RELATED APPLICATIONS

This application claims priority to U.S. Provisional Patent Application No. 62/993,648 titled “Analytical Toilet for Detecting Viruses in Feces” filed on 23 Mar. 2021, which is incorporated herein by reference in its entirety.

### TECHNICAL FIELD

The present disclosure relates to analytical toilets. More particularly, it relates to analytical toilets equipped to provide health and wellness information related to excreta deposited by a user.

### BACKGROUND

The ability to track an individual's health and wellness is currently limited due to the lack of available data related to personal health. Many diagnostic tools are based on examination and testing of excreta, but the high cost of frequent doctor's visits and/or scans make these options available only on a very limited and infrequent basis. Thus, they are not widely available to people interested in tracking their own personal wellbeing.

Toilets present a fertile environment for locating a variety of useful sensors to detect, analyze, and track trends for multiple health conditions. Locating sensors in such a location allows for passive observation and tracking on a regular basis of daily visits without the necessity of visiting a medical clinic for collection of samples and data. Monitoring trends over time of health conditions supports continual wellness monitoring and maintenance rather than waiting for symptoms to appear and become severe enough to motivate a person to seek care. At that point, preventative care may be eliminated as an option leaving only more intrusive and potentially less effective curative treatments. An ounce of prevention is worth a pound of cure.

One particular variety of detection, analysis, and trend tracking is related to biomarkers. “A bio-marker, or biological marker is a measurable indicator of some biological state or condition. Biomarkers are often measured and evaluated to examine normal biological processes, pathogenic processes, or pharmacologic responses to a therapeutic intervention. Biomarkers are used in many scientific fields.” (See https://en.wikipedia.org/wiki/Biomarker). Biomarker information can be a valuable resource in providing for the health and wellness of an individual or population. Some of the uses include being used to detect a disease at its earliest stages and monitoring the progression of key health metrics over time.

There can be significant risk to the health care professional whose responsibility it is to collect a sample for biomarker testing from a patient. The degree of risk depends on what biomarker is being tested for and which bodily fluid must be collected to test for the biomarker. For example, patients suspected of suffering from infection by a coronavirus have their throat swabbed or they provide a sample of sputum. This can require a health care professional to be in close proximity to the infected patient. A toilet equipped with a system to detect coronavirus can be located in a secure and isolated location where a patient can enter the location, leave a deposit in the toilet, depart the location, and not come into close contact with the health care professional.

### SUMMARY

In a first aspect, the disclosure provides an analytical toilet comprising a bowl to receive excreta and a detection system. The detection system includes a component functionalized with a probe or primer molecule to bind to the genetic material of a virus, such as a coronavirus. The toilet presents to the component a sample extracted from feces. The component creates a distinct signal indicating if a virus, such as a coronavirus, is present in the sample.

In a second aspect, the disclosure provides additional information related to the detection system, including the use of polymerase chain reaction to enhance the detection system and increase detection sensitivity.

Further aspects and embodiments are provided in the foregoing drawings, detailed description and claims.

### BRIEF DESCRIPTION OF THE DRAWINGS

The following drawings are provided to illustrate certain embodiments described herein. The drawings are merely illustrative and are not intended to limit the scope of claimed inventions and are not intended to show every potential feature or embodiment of the claimed inventions. The drawings are not necessarily drawn to scale; in some instances, certain elements of the drawing may be enlarged with respect to other elements of the drawing for purposes of illustration.

FIG. 1 illustrates an analytical toilet with the lid closed, according to an embodiment of the disclosure.

FIG. 2 illustrates an analytical toilet with lid open, according to an embodiment of the disclosure.

FIG. 3 illustrates an analytical toilet with lid closed and a portion of the exterior shell removed, according to an embodiment of the disclosure.

FIG. 4 further illustrates the interior of the toilet of FIGS. 1-3, according to an embodiment of the disclosure.

FIG. 5 illustrates a modular analytical test device attached to a manifold, according to an embodiment of the disclosure.

FIG. 6 illustrates another embodiment of a modular analytical test device.

FIG. 7 illustrates another embodiment of a modular analytical test device.

FIG. 8 illustrates an analytical test device adapted for use with a microfluidic chip, according to an embodiment of the disclosure.

FIG. 9 illustrates an MFC analytic test device with an optical interface, according to an embodiment of the disclosure.

FIG. 10 illustrates an MFC analytic test device with a set of electrical contacts, according to an embodiment of the disclosure.

FIG. 11 illustrates an alternate configuration for a larger MFC analytic test device with additional standardized areas designated for fluidic, electrical, or optical interconnects, according to an embodiment of the disclosure.

FIG. 12 illustrates the various steps of a method to detect a coronavirus in an analytical toilet, according to an embodiment of the disclosure, according to an embodiment of the disclosure.

### DETAILED DESCRIPTION

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

### Definitions

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure and are not meant to be limiting in any fashion. Nor do these phrases indicate any kind of preference for the disclosed embodiment.

As used herein, “toilet” is meant to refer to any device or system for receiving human excreta, including urinals.

As used herein, the term “bowl” refers to the portion of a toilet that is designed to receive excreta.

As used herein, the term “base” refers to the portion of the toilet below and around the bowl supporting it.

As used herein, the term “user” refers to any individual who interacts with the toilet and deposits excreta therein.

As used herein, the term “excreta” refers to any substance released from the body including urine, feces, menstrual discharge, and anything contained or excreted therewith.

As used herein, the term “sputum” refers to a mixture of saliva and mucus coughed up from the respiratory tract, typically as a result of infection or other disease and often examined to aid medical diagnosis.

As used herein, the term “manifold” is intended to have a relatively broad meaning, referring to a device with multiple conduits and valves to controllably distribute fluids, namely water, liquid sample and air.

As used herein, the term “test chamber” is meant to refer broadly to any space adapted to receive a sample for testing, receive any other substances used in a test, and apparatus for conducting a test, including any flow channel for a fluid being tested or used for testing.

As used herein, the term “sensor” is meant to refer to any device for detecting and/or measuring a property of a person or substance regardless of how that property is detected or measured, including the absence of a target molecule or characteristic.

As used herein, the term “microfluidics” is meant to refer to the manipulation of fluids that are contained to small scale, typically sub-millimeter channels. The “micro” used with this term and others in describing this invention is not intended to set a maximum or a minimum size for the channels or volumes.

As used herein, the term “microfluidic chip (MFC)” is meant to refer to is a set of channels, typically less than 1 mm², that are etched, machined, 3D printed, or molded into a microchip. The micro-channels are used to manipulate microfluidic flows into, within, and out of the microfluidic chip.

As used herein, the term “microfluidic chamber” is meant to refer to a test chamber adapted to receive microfluidic flows and/or a test chamber on a microfluidic chip.

As used herein, the term “lab-on-chip” is meant to refer to a device that integrates one or more laboratory functions or tests on a single integrated circuit. Lab-on-a-chip devices are a subset of microelectromechanical systems (MEMS) and are sometimes called “micro total analysis systems” (μTAS).

As used herein, the term “data connection” and similar terms are meant to refer to any wired or wireless means of transmitting analog or digital data and a data connection may refer to a connection within a toilet system or with devices outside the toilet.

As used herein, “biomarker” and “biological marker” are meant to refer to a measurable indicator of some biological state or condition, such as a normal biological processes, pathogenic processes, or pharmacologic responses to a therapeutic intervention. Some biomarkers are related to individual states or conditions. Other biomarkers are related to groups or classifications or states or conditions. For example, a biomarker may be symptomatic of a single disease or of a group of similar diseases that create the same biomarker.

As used herein, “analyte” is meant to refer to a substance whose chemical constituents are being identified and measured.

As used herein, the prefix “nano-” is meant to refer to something in size such that units are often converted to the nano-scale for ease before a value is provided. For example, the dimensions of a molecule may be given in nanometers rather than in meters.

As used herein, “miniaturized electronic system” is meant to refer to an electronic system that uses nanometer scale technology.

As used herein, “PCR” is meant to refer to polymerase chain reaction, which is a method widely used in molecular biology to rapidly make millions to billions of copies of a specific DNA sample allowing scientists to take a very small sample of DNA and amplify it to a large enough amount to detect and study in detail.

As used herein, “bind” and similar variants are meant to refer to the property of facilitating molecular interaction with a molecule, such as interaction with a molecular biomarker.

As used herein, “functionalize” and similar variants are meant to refer to a device, especially a nanometer scale device, the surface of which being configured to interact with a specific analyte, such as a specific biomarker.

As used herein, “genomic derived signal” is meant to refer to a molecule generated by the genome of a cell, bacteria, virus, or other nucleic acid carrier, such as DNA, RNA, microRNA, cell-free (circulating) nucleic acids or those of various immunologically related cells.

As used herein, a “fluidic circuit” is meant to refer to the purposeful control of the flow of a fluid. Often, this is accomplished through physical structures that direct the fluid flow. Sometimes, a fluidic circuit does not include moving parts.

As used herein, a “fluidic chip” is meant to refer to a physical device that houses a fluidic circuit. Often, a fluidic chip facilitates the fluid connection of the fluidic circuit to a body of fluid.

As used herein, “FET” is meant to refer to a field effect transistor, which is a device which uses an electric field to control the current flowing through a device. FETs are also known by the name “unipolar transistor”.

As used herein, a “primer” is meant to refer to a short single strand deoxyribonucleic acid (DNA) fragment known as an oligonucleotide that is a complementary sequence to the target DNA region of a biomarker. Living organisms use solely ribonucleic acid (RNA) primers, while laboratory techniques in biochemistry and molecular biology that require in vitro DNA synthesis (such as DNA sequencing and PCR) usually use DNA primers, since they are more temperature stable.

As used herein, a “NAT” is meant to refer to a nucleic-acid test which is a technique used to detect a particular nucleic acid sequence and thus usually to detect and identify a particular species or subspecies of organism, often a virus or bacteria that acts as a pathogen in blood, tissue, urine, etc. NATs differ from other tests in that they detect genetic materials (RNA or DNA) rather than antigens or antibodies. Detection of genetic materials allows an early diagnosis of a disease because the detection of antigens and/or antibodies requires time for them to start appearing in the stool, bloodstream, or other locations.

As used herein, a “NAAT” is meant to refer to a nucleic-acid amplification test to identify small amounts of DNA or RNA in test samples. They can, therefore, be used to identify bacteria, viruses, and other pathogens even when the material of interest is present in very small amounts. NAATs required an additional step to amplify the genetic material by making copies of it. NAATs are typically used in conjunction with such amplification methods as PCR, strand displacement assay (SDA), or transcription mediated assay (TMA).

As used herein, a “fluorescent tag” is meant to refer to a molecule that is attached chemically to aid in the detection of a biomolecule such as a protein, nucleic acid, antibody, or amino acid. Generally, fluorescent tagging, or labeling, uses a reactive derivative of a fluorescent molecule known as a fluorophore. The fluorophore selectively binds to a specific region or functional group on the target molecule and can be attached chemically or biologically.

As used herein, a “quantitative polymerase chain reaction instrument” is meant to refer to a machine that amplifies and detects DNA. It combines the functions of a thermal cycler and a fluorimeter, enabling the process of quantitative PCR.

As used herein, the term “virus” is given its ordinary meaning, namely a small infectious agent, comprised of genetic material within a capsid (protein coat), that replicates only inside the living cells of an organism.

As used herein, a “coronavirus” is a type of virus that causes diseases in birds and mammals. In humans, coronaviruses cause respiratory tract infections that can be mild, such as some cases of the common cold, and others that can be lethal. The coronavirus class of viruses includes alphacoronavirus, betacoronavirus, gammacoronavirus, or deltacoronavirus. More specifically, these viruses include severe acute respiratory syndrome (SARS-CoV), SARS-CoV-2 (also known as COVID-19), and middle east respiratory syndrome (MERS-CoV).

### EXEMPLARY EMBODIMENTS

The present disclosure relates to analytical toilets with analytical tools (may also be referred to as a “smart toilet” or a “health and wellness” toilet) which detects, analyzes, and/or tracks the trends of analytes, such as biomarkers, of a user who deposits excreta into the toilet. More specifically, the toilet receives feces from a user, processes the feces in preparation for analysis, and brings a sample extracted from the feces (including processed feces) into a testing area for detection of a virus, such as a coronavirus. A detection system in an analytical toilet described herein may be able to detect one or more viruses from the coronavirus class including alphacoronoavirus, betacoronovirus, gammacoronavirus, or deltacoronavirus. More preferably, the viruses that may be detected include SARS-CoV-2 (also known as COVID-19) or SARS-CoV. Other viruses include influenza A (e.g., H1N1, H1N2), dengue and Zika. The detection area may comprise a NAT or a NAAT detection system. The circuitry component has been functionalized to interact with a specific biomarker, such as the genetic material of a coronavirus, on a molecular or atomic level. The circuitry component provides a data signal depending on whether the specific biomarker is present in the feces sample in contact with it. After the toilet has finished with the feces, the toilet purges the feces from the toilet in preparation for receiving a new sample of excreta. In some embodiments, sputum that is deposited (i.e., spitted, expectorated) in the toilet may also be tested for a virus, such as a coronavirus.

In accordance with the present disclosure, an analytical toilet that includes an infrastructure for multiple health and wellness analysis tools is provided. This provides a platform for the development of new analytical tools by interested scientists and companies. Newly developed tests and diagnostic tools may be readily adapted for use in a system having a consistent tool interface.

In various exemplary embodiments, the analytical toilet provides a fluid processing manifold that collects and routes samples from the toilet bowl to various scientific test devices and waste handling portals throughout the device.

In various exemplary embodiments, the analytical toilet provides multiple fluid sources via a manifold system. The manifold is adapted to connect to a plurality of analytic test devices adapted to receive fluids from the manifold. The manifold is designed to selectively provide a variety of different fluid flows to the analytical test device. These fluids may include, among others, excreta samples, sputum, buffer solutions, reagents, water, cleaners, biomarkers, dilution solutions, calibration solutions, and gases such as air or nitrogen. These fluids may be provided at different pressures and temperatures. The manifold and analytical test device are also adapted to include a fluid drain from the analytical test devices.

In various exemplary embodiments, the manifold system provides a standardized interface for analytical test devices to connect and receive all common supplies (e.g., excreta samples, sputum, flush water), data, and power. Common supplies may be supplied from within (e.g., reagents, cleaners) or without (e.g., water) the toilet system. The analytical test devices may be designed to receive some or all of the standardized flows. The analytical test devices may also include storage cells for their own unique supplies (e.g., test reagent).

In various exemplary embodiments, the manifold is adapted to direct fluids from one or more sources to one or more analytical test devices. The manifold and analytical test devices are designed such that analytical test devices can be attached to and detached from the manifold making them interchangeable based on the needs of the user. Different analytical test devices are designed to utilize different test methods and to test excreta samples for different constituents.

In various exemplary embodiments, the analytical toilet provides an electrical power connection and a data connection for the analytical test device. In a preferred embodiment, the electrical power and data connections use the same circuit. In various exemplary embodiments, the toilet is provided with pneumatic and/or hydraulic power to accommodate the analytical test devices.

In various exemplary embodiments, the analytical toilet platform performs various functions necessary to prepare samples for examination. These functions include, but are not limited to, liquidizing fecal samples, diluting or concentrating samples, large particle filtration, sample agitation, and adding reagents. Miniaturized mechanical emulsification chambers may be used for repeatable and sanitary stool preparation. Stool samples may also be liquefied using acoustic energy and/or pressurized water jets.

In various exemplary embodiments, the analytical toilet also provides, among other things, fluid transport, fluid metering, fluid valving, fluid mixing, separation, amplification, storage and release, and incubation. The analytical toilet also is equipped to provide cleansers, sanitizers, rinsing, and flushing of all parts of the system to prevent cross-contamination of samples. In some embodiments, the system produces electrolyzed water for cleaning.

In various exemplary embodiments, one layer of the fluidic manifold is dedicated to macro-scale mixing of fluids. Sample, diluents, and reagents are available as inputs to the mixers. The mixing chamber is placed in series with all other scientific test devices, allowing bulk mixed sample to be routed to anywhere from one to all stations (i.e., analytical test device interfaces) for analysis. Mixing may also occur in an analytical test device.

In various exemplary embodiments, samples are filtered for large particulates at the fluid ingress ports of the manifold. The fluid manifold uses a network of horizontal and vertical channels along with simple valves to route prepared stool samples to one of several scientific test devices located on the platform.

In various exemplary embodiments, the manifold is constructed using additive layers, and different layers can be customized for particular applications. Standard ports and layouts are used for interfacing with external components, such as pressure sources and flow sensors. In general, characteristic channel volumes at the bottom of the manifold stack are on the order of milliliters. At the top of the manifold stack is the microfluidic science device, which will interface simultaneously with multiple microfluidic chips using standardized layout and pressure seals.

In various exemplary embodiments, the analytic test devices are designed to perform one or more of a variety of laboratory tests. Any test that could be performed in a medical or laboratory setting may be implemented in an analytical test device. These tests may include measuring pulse, blood pressure, blood oxygenation, electrocardiography, body temperature, body weight, excreta content, excreta weight, excreta volume, excreta temperature, excreta density, excreta flow rate, and other health and wellness indicators.

In various exemplary embodiments, the system is adapted to work with a variety of actuation technologies that may be used in the analytical test devices. The system provides electronic and fluidic interconnects for various actuator technologies and supports OEM equipment. In a preferred embodiment, the system is adapted to work with actuator modules that can be attached to the sample delivery manifold and controlled by a central processor. The system platform supports an inlet and outlet for the pressure transducer that interfaces with the fluidic manifold, and electronic or pneumatic connections where required. The system supports a variety of macro- and microfluidic actuation technologies including, but not limited to, pneumatic driven, mechanical pumps (e.g., peristaltic), on-chip check-valve actuators (e.g., piezo-driven or magnetic), electroosmotic driven flow, vacuum pumps, and capillary or gravity driven flow (i.e., with open channels and vents).

One benefit of the present disclosure is the detection, monitoring, and tracking of a user's biomarkers without having any inconvenience aside from what they would otherwise do using the toilet. Without the present disclosure, among other things, people often have to manually collect samples of feces, use equipment they are less familiar with than a toilet, or wait longer for analysis and results. Each of these things can negatively impact a user's experience and/or the quality or accuracy of the results.

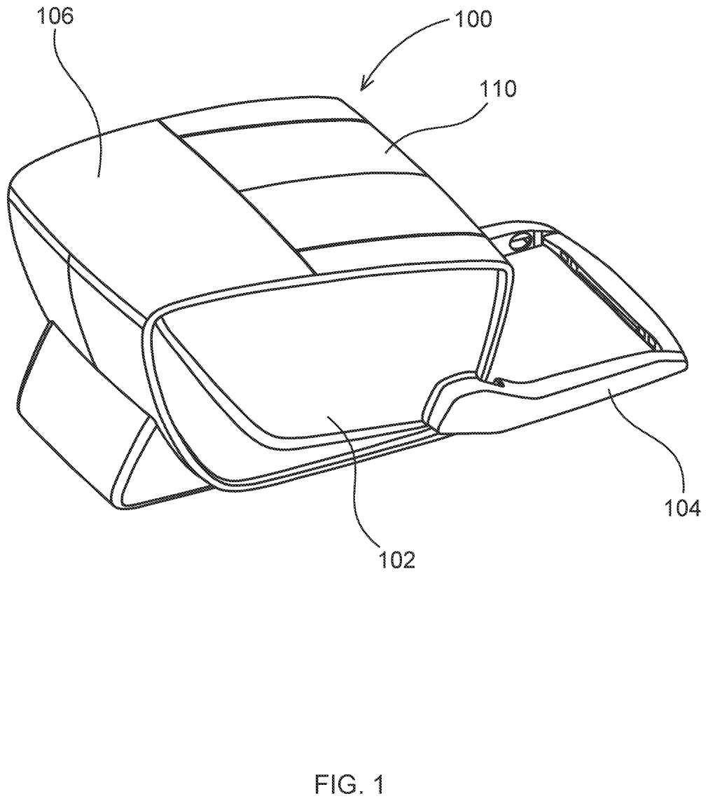

Now referring to FIGS. 1-3, a preferred embodiment of an analytical toilet 100 is shown. FIG. 1 illustrates the analytical toilet 100 with the lid 110 closed, according to an embodiment of the disclosure. FIG. 1 further shows exterior shell 102, foot platform 104 and rear cover 106. The lid 110 is closed to prevent a user from depositing feces or sputum in toilet 100 until the toilet is ready for use.

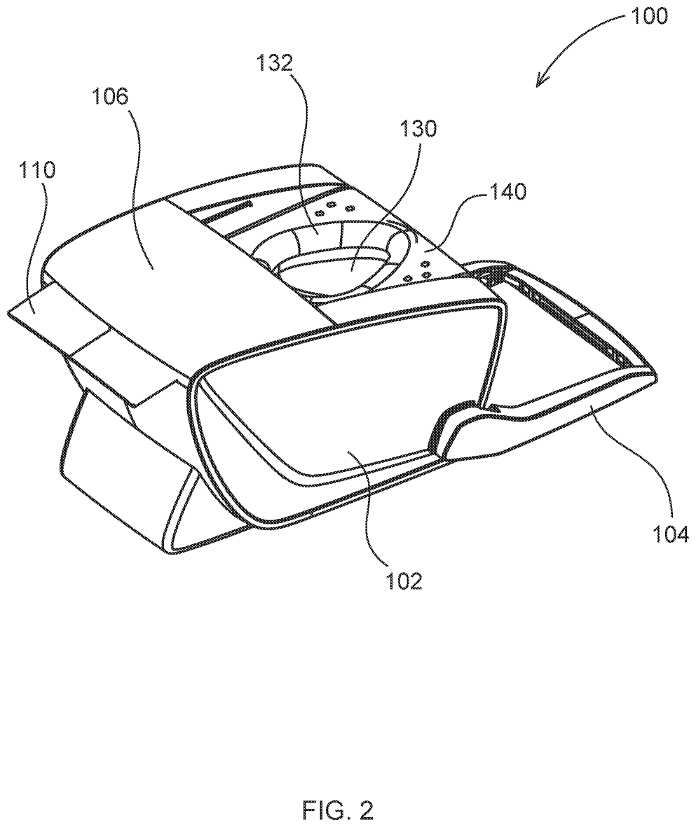

FIG. 2 illustrates toilet 100 with lid 110 open, according to an embodiment of the disclosure. Toilet 100 includes exterior shell 102, rear cover 106, bowl 130, seat 132, lid 110, fluid containers 140 and foot platform 104. Housed within toilet 100 are a variety of features, including equipment, that facilitate receiving excreta, processing feces or sputum for analysis, analyzing feces or sputum, and disposing of feces or sputum. FIG. 2 shows toilet 100 with lid 110 open so a user can sit on seat 132 and deposit feces in toilet 100.

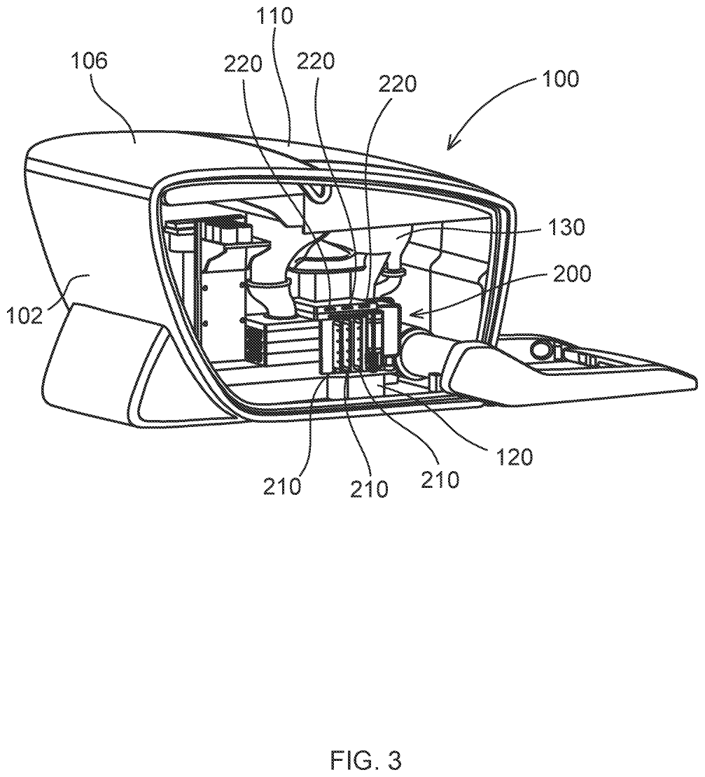

FIG. 3 illustrates toilet 100 with lid 110 closed and a portion of exterior shell 102 removed, according to an embodiment of the disclosure. This allows access to equipment housed within toilet 100. With exterior shell 102 removed, base 120 and manifold area 200 is visible. Manifold area 200 includes test areas 210 and fluidic chip slots 220. Preparation and/or analysis of sample can selectively take place in a test area 210 or fluidic chip slot 220. Manifold area 200 is the area where analysis takes place.

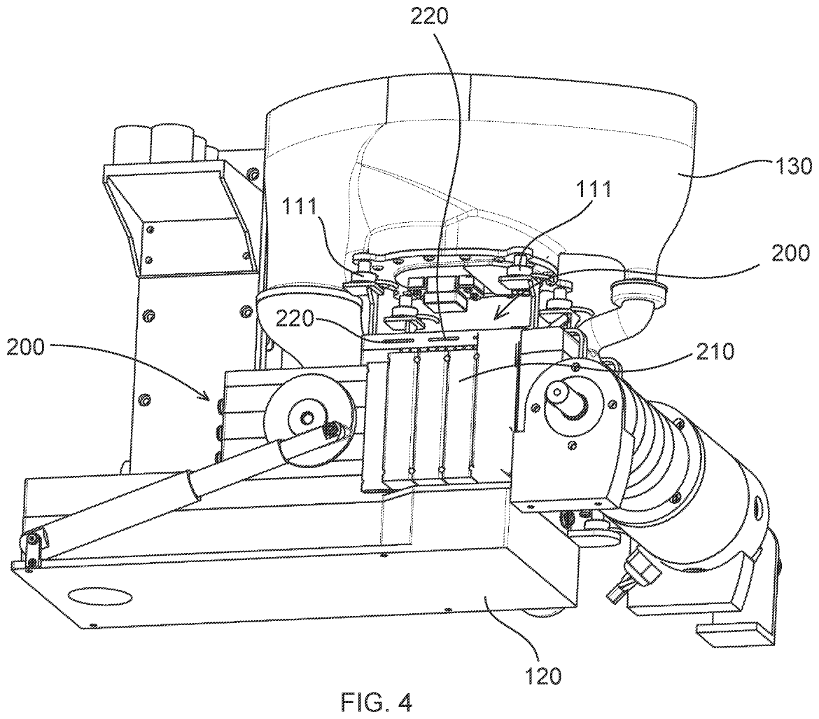

FIG. 4, further illustrates the interior of the toilet of FIGS. 1-3, according to an embodiment of the disclosure. The internal components of the toilet 100 are supported by a base 120. The bowl 130 is supported by one or more load cells 111. A manifold 200 is located below the bowl 130. The manifold 200 comprises a plurality of fluid paths. These fluid paths allow the manifold 200 to move fluids between the bowl 130, fluid containers 140, outside sources (e.g., municipal water supplies), other sources (e.g., air or water electrolyzing unit), analytical test devices 210, and the toilet outlet. The manifold 200 also provides electrical power and data connections to the analytical test devices 210. The manifold 200 can also directly pass fluids and/or solids from the bowl 130 to the toilet outlet.

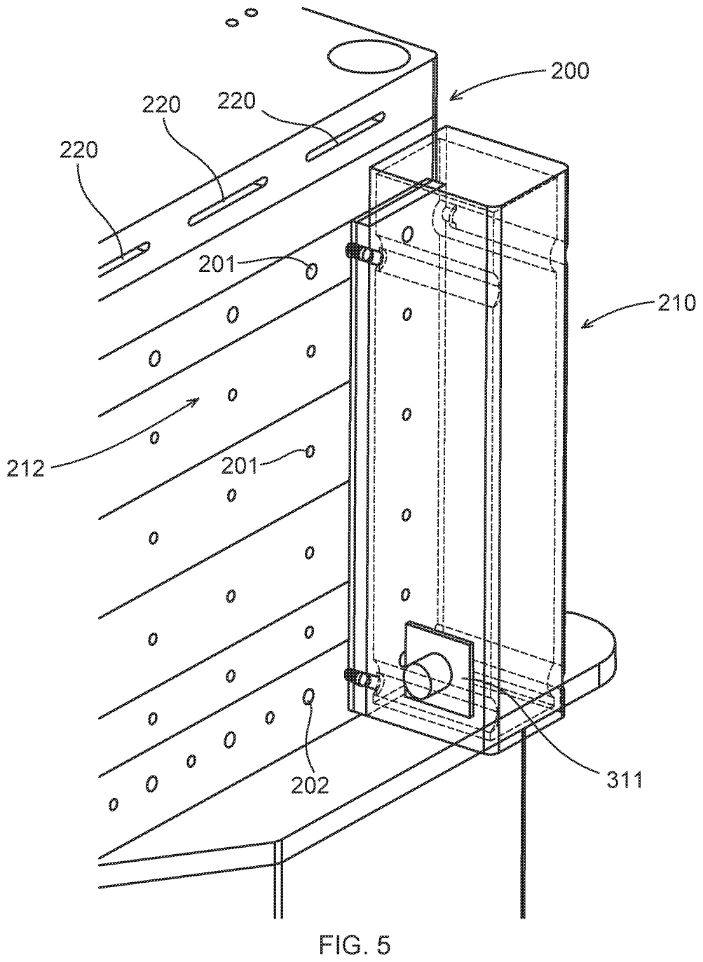

FIG. 5 illustrates a modular analytical test device 210 attached to a manifold 200, according to an embodiment of the disclosure. The manifold 200 is adapted to provide receptacles 210 with standardized connection interfaces for multiple analytical test devices 210. The manifold 200 is shown here with multiple fluid sources 201 for the analytical test device 210. In various embodiments, the manifold 200 may include receptacles 212 for more than one type of analytical test device 210 (e.g., different sizes, fluid supply needs, etc.). Slots 220 are also shown where microfluidic chips (MFCs) that further comprise detection system components may be inserted.

In various exemplary embodiment, the analytical test device 210 includes multiple inlets in fluid communication with the manifold 200. The selected fluid flows are directed into a test chamber with one or more detection systems 311 (flow channels internal to the analytical test device not shown in FIG. 5). The detection system 311 may comprise a NAT or NAAT device. In an exemplary embodiment, the sensor is a fluorimeter that measures changes in fluorescence of a sample comprising a fluorescent tag. The analytic test device 210 also includes at least one outlet 202 or drain in fluid communication with the manifold 200.

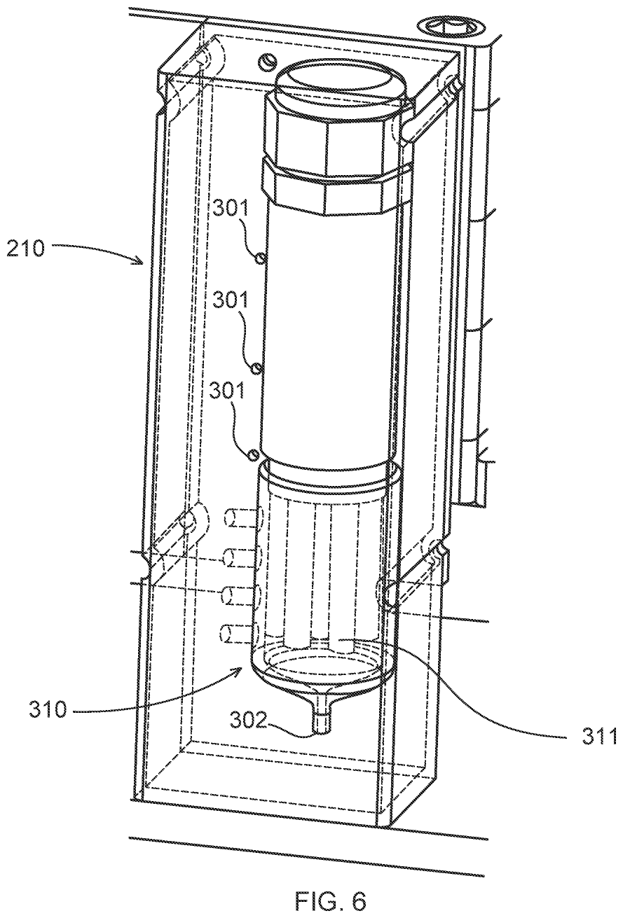

FIG. 6 illustrates another embodiment of a modular analytical test device 210. The analytical test device 210 includes multiple fluid inlets 301, test chamber 310, and at least one fluid outlet 302. The analytic test device 210 includes a test chamber 310 that received fluid flows and contains at least one detection system 311.

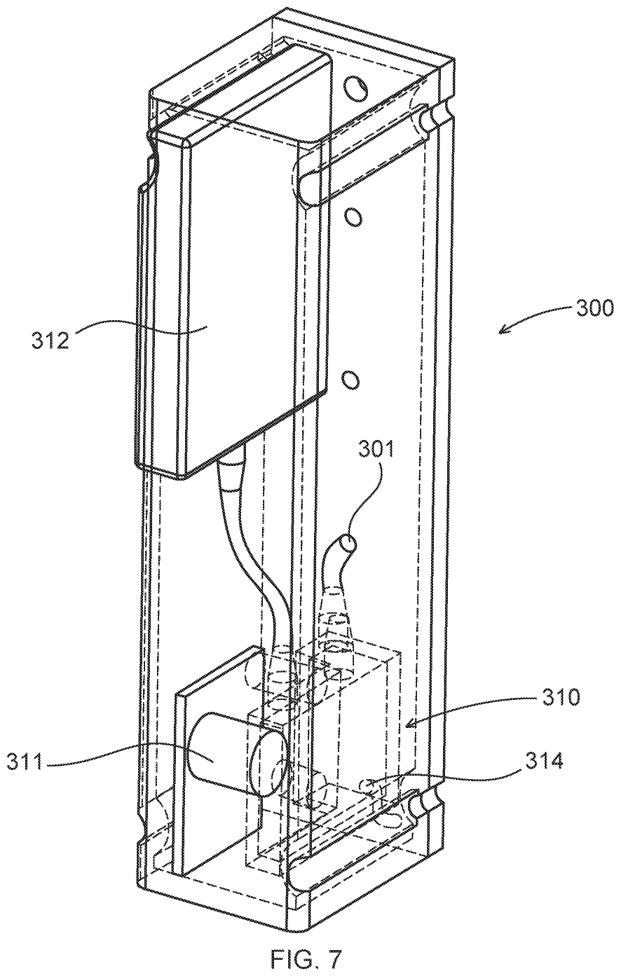

FIG. 7 illustrates another embodiment of a modular analytical test device 210. The analytical test device 210 includes multiple fluid inlets 301, test chamber 310, and at least one fluid outlet (not shown). This embodiment of an analytical test device 210 includes a storage cell 312, also in fluid communication with the test chamber 310. The analytical test device 210 may also include a pump to move fluid (e.g., test reagent) from the cell 312 to the test chamber 310. The analytic test device 210 also includes a camera adjacent to the test chamber 310 to monitor the contents of the test chamber 310.

There are many ways to incorporate the detection system into the toilet, the selection of which will depend on various factors, including ease of manufacture and maintenance, target market, physical constraints, frequency of use compared to other desired functions of the toilet, and cost. In one preferred embodiment, the detection system is integrated with a fluidic circuit. More preferably, the fluidic circuit is on a fluidic card. Still more preferably, the fluidic circuit on the fluidic card is a microfluidic circuit on a micro fluidic card. Yet more preferably, the microfluidic circuit interfaces with nano-scale fluidic circuits. Preferably, the fluidic card is inserted into a slot or receptacle of the toilet which connects the fluid circuit on the card to the toilet's fluidic delivery system, enabling the card to receive the sample derived from the feces. Alternatively, the detection system is part of a larger device that may be attached to the toilet, such as a device that processes and/or analyzes the sample extracted from the feces. Alternatively, the detection system is built into the toilet rather than being on a card. Alternatively, the sensor is external to the remainder of the toilet and is connected to receive and/or return fluid from the toilet, such as may be accomplished by connecting the sensor to part of the toilet with tubes or pipes.

Sample Extraction of Virus from Feces

Once feces has been deposited in the toilet, there are many ways it can be processed in preparation for virus testing and disposal. There are different challenges associated with extracting a sample for testing from feces as opposed to urine. Some pretreatments include comminution, filtration, centrifuging, washing, dilution, or pH normalization. In one preferred embodiment, a portion of feces is separated from urine. The feces is then washed, mixed with buffer, water, and/or a reagent, and presented to the component of a detection system for analysis. Following analysis, the sample is removed from the detection system, and the system is cleaned and/or sterilized in preparation for a new sample being presented to the component of the detection system. This method allows for extraction and detection of viruses, such as coronaviruses, found on the surface or mixed in with the feces.

In some instances, it may be necessary to use a more aggressive method to extract genetic material from the coronaviruses found in the feces. The feces may be processed in order to extract a portion of intracellular material contained within intact viruses within the feces. This allows for testing of the released genetic material. In particular, this allows for testing of free cell RNA or other intraviral material. The viral material may comprise material extracted from a coronavirus. The viruses may be burst or lysed open (also referred to as cytolysis) using a variety of methods. There are several methods that an analytical toilet may be developed to lyse material. Physical methods to lyse viruses include mechanical disruption, liquid homogenization, high frequency sound waves, freeze/thaw cycles, and manual grinding. Physical methods may induce local heating which may lead to protein denaturation and aggregation. The feces may need to be chilled in some instances. Viruses burst at different times, so subviral components may be subjected to ongoing disruptive forces which may lead to too much virus component degradation.

In some cases, solution-based viral lysis (also referred to as reagent-based viral lysis) may be preferred to release intraviral material of coronaviruses in an analytical toilet. Solution-based cell lysis tends to be a more rapid, gentle, efficient, and reproducible method, and leads to a high protein yield. Solution-based virus lysis can be used to extract total protein or subcellular fractions from various coronaviruses, such as RNA. Solution-based viral lysis works by disrupting the lipid membrane and/or virus wall. Solution-based lysis reagents may include detergents such as CHAPS (3-cholamidopropyl dimethylammonio 1-propanesulfonate) or the Triton-X series of nonionic detergents. Solution-based lysis reagents may include mammalian cell lysis reagents.

In one embodiment of a method to detect coronavirus in feces, a sample of feces may first be rinsed/washed with a fluid. A rinsing/washing step can be used to remove urine. Urine can comprise different analytes than feces that may interfere in testing for virus genetic material from feces. The fluid may be aqueous-based such as deionized water, tap water, distilled water or a buffer. The fluid may be an organic material such as methanol, ethanol, isopropanol or other hydrocarbon. The rinse fluid may then be disposed of down the drain or some other location. In some instances, the rinse fluid may be retained for further testing. The buffer may have a pH in the range of about 1-12. The pH of the buffer will vary depending on the analytes being tested for. For example, for a pH range of about 1-2.2, HCl/KCl may be used as a buffer. For a pH range of about 2.2-3.6, glycine/HCl may be used as a buffer. For a pH range of about 2.2-4.0, potassium hydrogen phthalate/HCl may be used as a buffer. For a pH range of about 3.0-6.2, citric acid/sodium citrate may be used as a buffer. For a pH range of about 3.7-5.6, sodium acetate/acetic acid may be used as a buffer. For a pH range of about 4.1-5.9, potassium hydrogen phthalate/NaOH may be used as a buffer. For a pH range of about 5.5-6.7, 2-(N-morpholino) ethanesulfonic acid (MES) may be used as a buffer. For a pH range of about 5.8-7.2, bis-tris methane (BIS-TRIS) may be used as a buffer. For a pH range of about 5.8-8.0, phosphate buffer (PBS) may be used as a buffer. For a pH range of about 6.0-7.2, N-(2-acetamido) iminodiacetic acid (ADA) may be used as a buffer. For a pH range of about 6.1-7.5, piperazine-N,N′-bis(2-ethanesulfonic acid) (PIPES) or N-2-aminoethanesulfonic acid (ACES) may be used as a buffer. For a pH range of about 6.2-7.6, 3-morpholino-2-hydroxypropanesulfonic acid (MOPSO) may be used as a buffer. For a pH range of about 6.3-9.5, 1,3-bis(tris(hydroxymethyl)methylamino)propane (BTP) may be used as a buffer. For a pH range of about 6.4-7.8, BES may be used as a buffer. For a pH range of about 6.5-7.9, 3-(N-morpholino)propanesulfonic acid (MOPS) may be used as a buffer. For a pH range of about 6.8-8.2, 2-[(2-Hydroxy-1,1-bis(hydroxymethyl)ethyl)amino]ethanesulfonic acid (TES) or 4-(2-hydroxyethyl)-1-piperazineethanesulfonic acid (HEPES) may be used as a buffer. For a pH range of about 6.9-8.3, 4-(N-morpholino)butanesulfonic acid (MOBS) may be used as a buffer. For a pH range of about 7.0-8.2, 3-(N,N-Bis[2-hydroxyethyl]amino)-2-hydroxypropanesulfonic acid (DIPSO) or 2-Hydroxy-3-[tris(hydroxymethyl)methylamino]-1-propanesulfonic acid (TAPSO) may be used as a buffer. For a pH range of about 7.0-9.0, 2-amino-2-(hydroxymethyl)-1,3-propanediol (Trizma base) may be used as a buffer. For a pH range of about 7.1-8.5, 4-(2-Hydroxyethyl)piperazine-1-(2-hydroxypropanesulfonic acid) Hydrate hydrate (HEPPSO) may be used as a buffer. For a pH range of about 7.2-8.5, piperazine-1,4-bis(2-hydroxypropanesulfonic acid) dihydrate (POPSO) may be used as a buffer. For a pH range of about 7.4-8.8, tricine may be used as a buffer. For a pH range of about 7.5-8.9, diglycine (Gly-Gly) may be used as a buffer. For a pH range of about 7.6-9.0, 2-(bis(2-hydroxyethyl)amino)acetic acid (BICINE) or N-(2-hydroxyethyl)piperazine-N′-(4-butanesulfonic acid) (HEPBS) may be used as a buffer. For a pH range of about 7.7-9.1, [tris(hydroxymethyl)methylamino]propanesulfonic acid (TAPS) may be used as a buffer. For a pH range of about 7.8-9.7, 2-Amino-2-methyl-1,3-propanediol (AMPD) or N-tris(Hydroxymethyl)methyl-4-aminobutanesulfonic acid (TABS) may be used as a buffer. For a pH range of about 8.3-9.7, N-(1,1-Dimethyl-2-hydroxyethyl)-3-amino-2-hydroxypropanesulfonic acid (AMPSO) may be used as a buffer. For a pH range of about 8.6-10.0, N-cyclohexyl-2-aminoethanesulfonic acid (CHES) may be used as a buffer. For a pH range of about 8.6-10.3, N-cyclohexyl-2-hydroxyl-3-aminopropanesulfonic acid (CAPSO) may be used as a buffer. For a pH range of about 9.0-10.5, 2-Amino-2-methyl-1-propanol (AMP) may be used as a buffer. For a pH range of about 9.7-11.1, N-cyclohexyl-3-aminopropanesulfonic acid (CAPS) may be used as a buffer. For a pH range of about 10.0-11.4, 4-(cyclohexylamino)-1-butanesulfonic acid (CABS) may be used as a buffer.

In the instances where the rinse fluid is tested, other additives may be added to the rinse fluid or in combination with one or more buffers in order to preserve any coronavirus genetic material. Such additives may be one or more reducing agents to protect the genetic material from damage due to oxidation. Reducing agents may include dithiothreiotol (DTT), 20mercaptoethanol (BME) or tris(2-carboxyethyl) phosphine (TCEP) or combinations thereof. Another additive may be a protease inhibitor to inhibit protein degradation such as phenylmethylsulfonyl fluoride (PMSF), 4-(2-aminoethyl) benzenesulfonyl fluoride hydrochloride (AEBSF), ethylenediaminetetraacetic acid (EDTA), ethylene glycol-bis (O-aminoethyl ether)-N,N,N′,N′-tetraacetic acid (EGTA), benzamidine, leupeptin, aprotinin, chymostatin, antipain or combinations thereof. Another additive may be an osmolyte to stabilize the coronavirus genetic material and improve solubility such as glycerol, detergent or a sugar or a combination thereof. Another additive may be an ionic stabilizer to enhance solubility such as a NaCl, KCl or (NH₄)₂SO₄ or a combination thereof. Another additive may be an α-helix stabilizer trifluoroethanol (TFE), trimethylamine N-oxide (TMAO) or a molecular specific chaperone such as a heat shock protein or ligand.

In one embodiment of a method to detect coronavirus in feces, a user first deposits feces into an analytical toilet. The analytical toilet removes a portion of the feces for further processing. The portion of feces may be rinsed with one or more fluids described previously. Alternatively, the portion of feces may not be rinsed. The rinsed or non-rinsed feces may first be exposed to a physical or reagent-based lysing method to release the intracellular material located within the feces. The lysed material may be nucleic acids from one or more viruses. Once the feces is treated with a lysing method, the lysed material may then be rinsed with a second round of fluids such as buffers, stabilizer, protease inhibitors or other fluids as previously described herein. This preserves the genetic material (i.e., biomaterial) and/or prepares it for detection of genetic material, such as virus RNA. The genetic material may be nucleic acids, such as RNA or DNA. The rinsed material may then be filtered and transported using a microfluidic system to one or more detection systems to analyze the fluid.

There are a variety of classes of virus-based biomarkers extracted from feces that may be detectable. For example, some groupings worth being considered are ionic or electrochemical, immunological, chromogenic, labeling or biotinylation, fluorescent binding, staining reactions, and transfection or genotypic. There are many variations on detection systems that detect biomarker molecules or atoms that may be used in a health and wellness analytical toilet described herein. It should be known that although the disclosure herein describes extraction of analytes, such as biomarkers, from feces, detection of coronaviruses may also be carried out from urine simultaneously or in series. Health and wellness analytical toilet embodiments described herein may comprise one or more detection systems to detect coronaviruses in feces and one or more detection systems to detect coronaviruses found in urine.

### NAT-Based Detection System

Once the viral genetic material is released, isolated, and purified from the feces, the genetic material can then be tested for coronavirus. One exemplary class of coronavirus detection systems in an analytical toilet are NATs and NAATs. NAATs are typically preferred over NATs as NAATs have higher sensitivity. The NAAT-based detection system in an analytical toilet may comprise nucleic acid extraction, purification, amplification, detection, and data analysis steps. The NAAT system may be used to detect unique sequences of coronavirus RNA. The genetic material of a coronavirus is made up of RNA. The NAAT system may be used to detect coronaviruses by targeting N, E, S and RdRP (RNA-dependent RNA polymerase) genes in the virus.

A next step in detecting for coronavirus in isolated and purified genetic material from fecal material is to amplify the genetic material to increase sensitivity and make it easier to analyze the genetic material. The amplification process may be completed in a chamber. An analytical toilet for detecting a coronavirus may comprise amplification methods of strand displacement assay (SDA), loop mediated isothermal amplification (LAMP), quantitative nucleic acid sequence-based amplification (QT-NASBA) or transcription mediated assay (TMA). In an exemplary embodiment, an analytical toilet for detecting a coronavirus may comprise the amplification method of polymerase chain reaction (PCR). Other PCR-related amplification methods that may be used in an analytical toilet for detection of a coronavirus include allele-specific PCR, assembly PCR, asymmetric PCR, convective PCR, dial-out PCR, digital PCR, helicase-dependent amplification, hot start PCR, in silico PCR, intersequence-specific PCR (ISSR), inverse PCR, ligation-mediated PCR, methylation-specific PCR (MSP), miniprimer PCR, multiplex ligation-dependent probe amplification (MLPA), multiplex-PCR, nanoparticle-assisted PCR (nanoPCR), nested PCR, overlap-extension PCR, quantitative PCR (qPCR), reverse transcriptase PCR (RT-PCR), RNase H-dependent PCR (rhPCR), single specific primer-PCR (SSP-PCR), solid phase PCR, suicide PCR, thermal asymmetric interlaced PCR (TAIL-PCR), touchdown PCR (Step-down PCR) or universal fast walking (UFW).

It should be noted that PCR methods of amplification are used to replicate copies of DNA samples. The genetic material in coronaviruses is RNA. In order to detect coronavirus RNA using PCR in an analytical toilet, the method of RT-PCR may be used. RT-PCR uses messenger RNA (mRNA) rather than DNA as the starting template, amplifying complementary DNA (cDNA). First, the enzyme reverse transcriptase uses the mRNA template to produce a complementary single-stranded DNA strand called cDNA in a process known as reverse transcription. Next, DNA polymerase is used to convert the single-stranded cDNA into double-stranded DNA. These DNA molecules can now be used as templates for a PCR reaction as described above. The value of RT-PCR is that it can be used to determine if an mRNA species is present in a sample, such as coronavirus RNA.

Various components and reagents that may be needed in an analytical toilet to carry out amplification of coronavirus genetic material includes a mRNA template, reverse transcriptase enzyme, DNA polymerase, DNA primers, deoxynucleotide triphosphates (dNTPs), a buffer solution that allows for optimal stability and activity of the DNA polymerase, bivalent cations such as Mn²⁺ or Mg²⁺ and monovalent cations such as K⁺ or Na⁺. Various inlet and outlet passageways may be integrated with the chamber where reagents may be injected into the chamber and reaction products may be transported out of the chamber.

Various procedures that may be needed in an analytical toilet to carry out amplification of coronavirus genetic material includes initialization, denaturation, annealing, extension/elongation. These steps constitute a single thermal cycle. Two or more cycles may be needed to amplify the coronavirus genetic material sufficiently for detection and analysis. Thermal cycles at higher temperatures are needed for denaturation while lower temperatures are needed for annealing. Further steps include final elongation and final hold.

An analytical toilet may be equipped with a quantitative PCR (qPCR) instrument. The qPCR instrument may be used to carry out the amplification and detection of the genetic material of one or more coronaviruses. The detection method may use fluorescence spectroscopy. Fluorescent tags are covalently or ionically bound to the primer molecules to monitor amplification and detection of coronavirus RNA using a fluorimeter. One or more of the following fluorescent tags may be used: 6-FAM™, JOE™, TET™, Cal Fluor Gold 540, HEX™, Cal Fluor Orange 560, TAMRA™, Cyanine 3, Quasar™ 570, Cal Fluor Red 590, ROX™, Texas Red™, Cyanine 5, Quasar™ 670, Cyanine 5.5, NED, Cal Fluor Red 610 or 635, VIC, Bioresearch Blue™, Pulsar™ 650, Quasar™ 705 or a combination thereof. The fluorescent tags emit in different detectable wavelengths. When a hybridization event occurs between a primer molecule and an amplification product, a distinct change in fluorescence is detected by a fluorimeter.

The fluorescent tags may be matched with a quencher such as Biosearch's Black Hole Quencher (BHQ) dyes. Quencher dyes provide spectral overlap over the entire range of commonly used reporter dyes. Dyes typically cover the spectrum from 430 nm into the near IR making it possible to utilize reporter dyes that emit anywhere in this range. Dyes have no fluorescence of their own. Fluorescence is not seen until a hybridization event occurs.

In some embodiments, a qPCR instrument may be combined with a FET-based sensor in an analytical toilet. FET-based sensors comprise a bridge component that bridges the source and drain electrodes. Bridge components may comprise an electrically conducting nanowire such as carbon nanotube or a conducting polymer. Small perturbations in the bridge component causes distinct and detectable changes in the current between the source and drain. Perturbations may be caused by interaction of the bridge component with an analyte such as a biomarker. The FET-based sensor may comprise a primer, aptamer, oligonucleotide or other element to interact with a component of a virus, such as coronavirus RNA. A qPCR instrument may amplify the virus genetic material extracted from feces or sputum in order to be detected by a FET-based sensor.

A coronavirus detection system in an analytical toilet may comprise a paper-based NAAT. The paper-based NAAT may be combined with a microfluidic system. The paper-based NAAT may comprise a lateral flow assay (LFA), lateral flow immunoassay (LFIA), micropaper analytical device (microPAD), or a two-dimensional paper network (2DPN). The paper-based NAAT may use an isothermal amplification process such as loop-mediated isothermal amplification (LAMP), helicase-dependent amplification (HDA), rolling circle amplification (RCA), recombinase polymerase amplification (RPA), or isothermal strand displacement amplification (iSDA). Other isothermal paper-based NAATs that may be used in an analytical toilet to detect a coronavirus include nucleic acid sequence-based amplification (NASBA), rolling circle amplification (RCA), signal-mediated amplification of RNA technology (SMART), or nicking endonuclease signal amplification (NESA)/nicking endonuclease assisted nano-particle activation (NEANA). In some embodiments, a paper-based NAAT in an analytical toilet may comprise the clustered regularly interspaced short palindromic repeat protein Cas12. An analytical toilet described herein may incorporate the DETECTR system (Mammoth Biosciences, South San Francisco, Calif., USA) to detect viruses, such as the coronavirus class of viruses.

A detection system in an analytical toilet described herein may also be able to detect polysaccharides that are extracted from a sample of feces. Detection of C-polysaccharide can be useful in determining pneumonia, respiratory infections, treatment effectiveness.

In some embodiments, NAT and NAAT-based detection systems in a health and wellness analytical toilet may be combined with other methods of coronavirus biomarker detection. Additional biomarkers may be measured via a miniaturized mass spectrometer. Alternatively, additional biomarkers may be measured using gas chromatography integrated into the toilet body or positioned adjacent to the toilet.

In various exemplary embodiments, microfluidic systems may be used to isolate and transport a sample, add, and mix reagents if appropriate, filter out solids, and test the sample for one or more coronaviruses on a small scale (i.e., sub-millimeter scale) in a health and wellness analytical toilet described herein. The microfluidic system may comprise an open microfluidic system, continuous-flow microfluidic system, droplet-based microfluidic system, digital microfluidic system, nanofluidic system, paper-based microfluidic system or combinations thereof.

A microfluidic-based coronavirus detection system may be located on a microfluidic chip (MFC). In a preferred embodiment, the MFC includes a test chamber with a lab-on-chip (“LoC”) (also known as “test-on-chip”). The LoC may be designed to perform one or more laboratory tests. In various exemplary embodiments, one or more microfluidic chips (MFCs) may be removed or added to the toilet system as desired or needed at any given time, such as for different biomarker tests. In an exemplary embodiment, a DNA microfluidic chip may be used as a component in a biomarker sensor in a health and wellness analytical toilet. The DNA chip may comprise a DNA microarray, such as the GeneChip DNAarray (Affymetrix, Santa Clara, Calif., USA). The DNA microarray comprises one or more pieces of DNA (probes) for biomarker detection. The MFC may comprise one or more affixed proteins in an array-like fashion. In an exemplary embodiment, the proteins are monoclonal antibodies for detection of antigens.

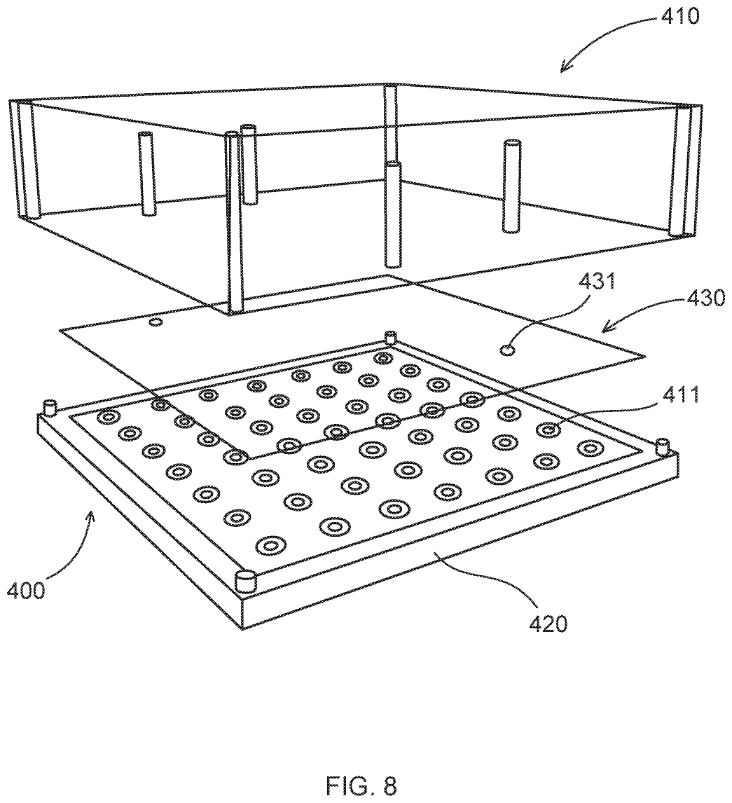

FIG. 8 illustrates an analytical test device 400 adapted for use with a microfluidic chip (MFC), according to an embodiment of the disclosure. The MFC analytical test device 400 includes a test chamber 410 with a lab on chip 411. The LoC 411 may comprise one or more detection system components previously described herein. The manifold 200 includes a plurality of slots 220 or other openings for placement of a MFC analytical test device 400. An interface 420 with multiple ports 421, which act as fluid inlets or outlets for the test chamber 410, to provide connections and/or supplies for the MFC analytic test device 400. Protrusions 422 encircle the ports 421 to provide a point of positive contact for the sealing gasket 430, minimizing dead volume. Pores 431 in the gasket 430 select or block possible interactions with the MFC analytical test device 400.

In various exemplary embodiments, the backplate interface 420 is machined or molded with multiple microfluidic pores 421 for ingress of fluids or for removal of fluids. The interface 420 can be used with a variety of MFC analytic test device 400 testing modules. The ports 421 are preferably sealed by placing a gasket 430 between the MFC analytic test device 400 and backplate 420. The gasket 430 may be designed with pores 431 to selectively allow fluid flow through selected ports 421 and block potential flow through others depending on the MFC analytic test device 400 design. The gasket 430 may have alignment holes or features, with matching structures in the interface 420 or MFC analytic test device 400, to facilitate aligning the sheet of material to the pores 431. For example, the gasket 430 may fit snugly in a recess in the interface 420, or alignment pins in the interface 420 may match holes patterned in the gasket 430 by the same process used to create the pores 431. The gasket 430 may have corrugations, or variations in thickness, or be composed of multiple layers of different materials designed to reduce distortions propagating from one point of contact to another, in order to improve the alignment of the gasket 430 to the pores 431 during installation or when the system is pressurized.

In various exemplary embodiments, the gasket 430 may function as a seal for otherwise open channels in the MFC analytic test device 400, permitting pressurized flow in those channels. The gasket 430, typically an electrical insulator, may have electrical contacts built into the top, bottom, or intermediate layers of material, or embedded within the gasket 430 material. The gasket 430 may have electrical contacts built to cause a voltage potential to exist in the fluid. The gasket 430 may have electrical contacts built on the surface to come into contact with the fluid or gas and provide a potential to create electrochemical interactions. The gasket 430 may have electrical contacts built on the surface to come into contact with the fluid or gas and measure the electrical potential or ionic current.

In various exemplary embodiments, when the MFC analytic test device 400 is mechanically clamped to the plate 420 with sufficient pressure, the gasket 230 material creates a high-pressure seal between the MFC analytic test device 400 and the interface 420. Pores 431 in the gasket 430 open a channel between the backplate 420 and the MFC analytic test device 400. The gasket 430 material may be optimized for the application, including selecting chemically inert, chemically stable, bio-compatible, or anti-microbial material. Alternatively, each pore 431 may be circumscribed with an elastomer O-ring that provides a seal under pressure.

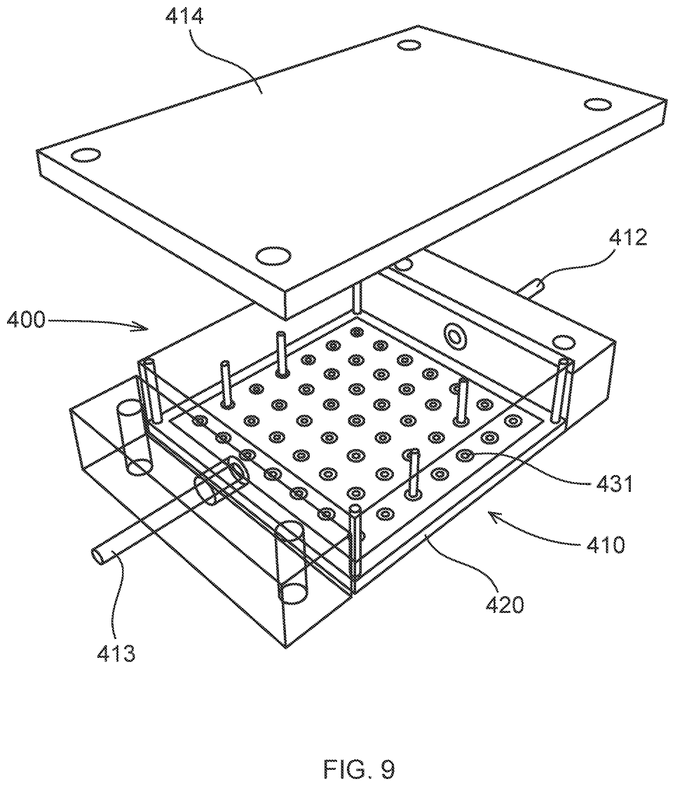

FIG. 9 illustrates an MFC analytic test device 400 with an optical interface, according to an embodiment of the disclosure. In some embodiments, a light source and light detector are connected to the test chamber 410 by fiber optic cables 412 and 413 are part of the test chamber 410 (e.g., spectrometer). The MFC analytical test device 400 is attached to the interface and held in place by a clamp plate. In an alternative embodiment, the light source 412 may be placed in the clamp plate 414 to deliver light to a standardized location normal to the MFC analytic test device 400.

In various exemplary embodiments, the light detector may include various mechanisms for reducing reflections, such as covering the detector with an anti-reflection coating, film. The light detector may be an optical waveguide or other photonic sink. The light detector may include various lenses for amplifying or collimating light to the detector, or for efficiently coupling collected light into the waveguide.

In various exemplary embodiments, the MFC analytical test device 400 is secured to the interface 420 by a clamp 414. In a preferred embodiment, the clamp 414 serves a dual purpose as a back-side interface for microfluidic ports 421 in the reverse side of the MFC analytic test device 400. Microchannels machined into the clamp 414 in standardized locations may be included in the clamp design. The clamp can be mechanically actuated so that the chip or its replacement may be easily and repeatedly inserted and removed.

In various exemplary embodiments, the microfluidic fixtures, including screw-in connectors, may interface with micro-tubing to provide a connection elsewhere in the system, or back to the same chip. The micro-tubing may provide chip-to-chip connections.

In various exemplary embodiments, the clamp may serve as a housing or mounting point for a mirror that directs laser light through the MFC analytic test device 400. Some implementations may use a semi-transparent mirror that allows several MFC analytic test devices 400 arranged in a line to use the same laser beam to interact with MFC analytic test device 400 components. The platform microfluidic interface/manifold provides ports intended for this purpose.

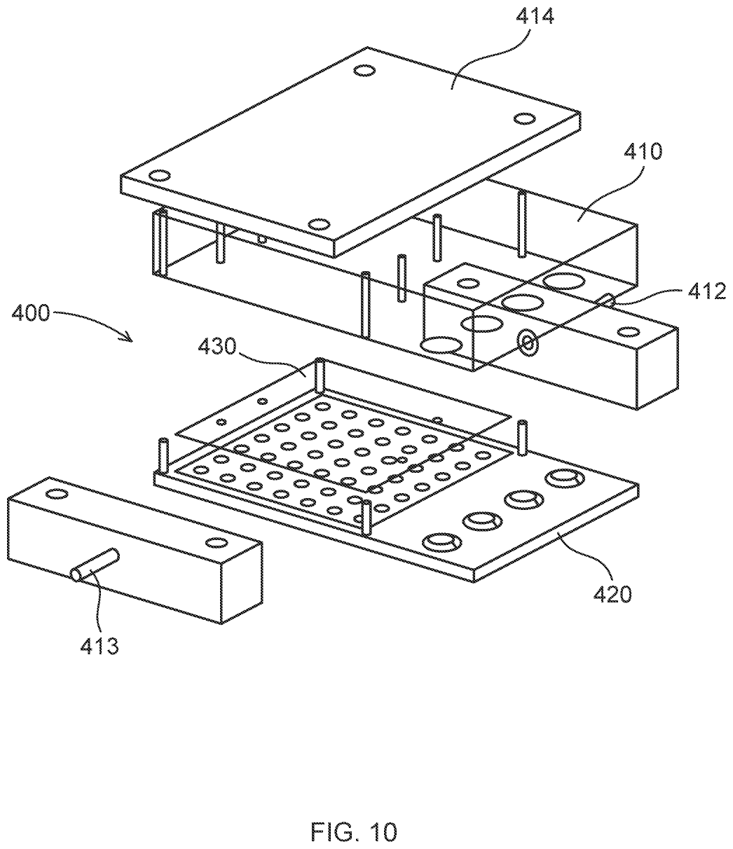

FIG. 10 illustrates an MFC analytic test device 400 with a set of electrical contacts, according to an embodiment of the disclosure. Electrical pads in a standardized location are made available for generic electrical operations, such as providing high and low voltage contacts, control signals, and ground pins. Corresponding pads are also located on the MFC analytic test device 400. The electrical contacts may be constructed with mechanical compliance within a recess, permitting chamber 410 and interface 420 to remain parallel when in contact.

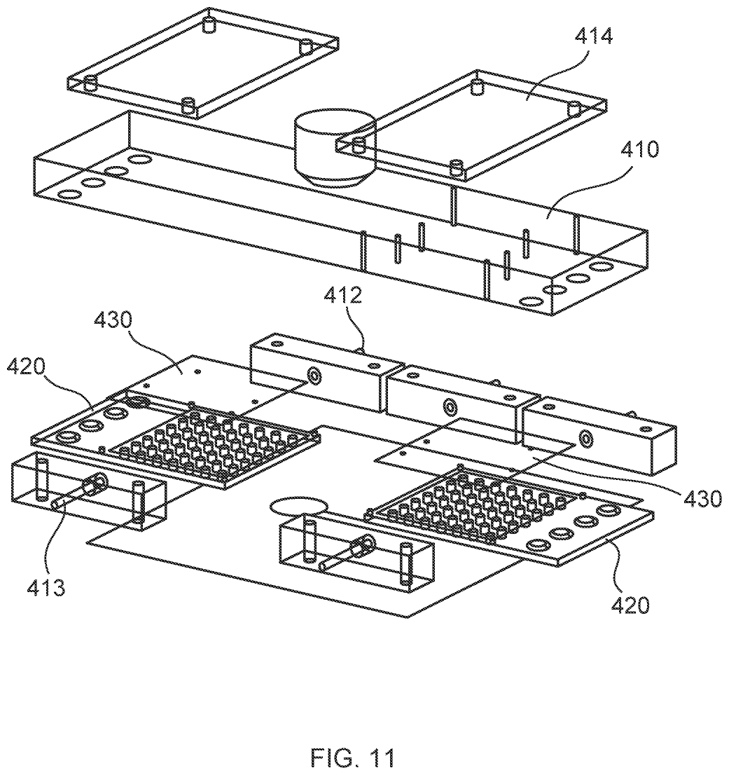

FIG. 11 illustrates an alternate configuration for a larger MFC analytic test device 400 with additional standardized areas designated for fluidic, electrical, or optical interconnects, according to an embodiment of the disclosure. This embodiment shows a designated viewing area, where a light source illuminates the MFC analytic test device 400 from below, and an image detector (possibly including a microscope or other magnifier) examines the output. Images may be stored for post-processing. Other embodiments may include an electronically controlled shutter that selectively blocks the light or selects a pinhole/orifice size for the light. The light source may be visible, UV, or other wavelength range of light emissions. The light source may be wide-band or narrow band.

In various exemplary embodiments, the MFC analytical test device 400 is designed to use very small quantities of reagent. In various exemplary embodiments, reagents are dispensed using technology similar to that used in inkjet printers to dispense ink. In some embodiments, an electrical current is applied piezoelectric crystal causing its shape or size to change forcing a droplet of reagent to be ejected through a nozzle. In some embodiments, an electrical current is applied to a heating element (i.e., resistor) causing reagent to be heated into a tiny gas bubble increasing pressure in the reagent vessel forcing a droplet of reagent to be ejected.

In various exemplary embodiments, the toilet fluidic manifold provides routing. Interconnecting levels of channels allows routing from one port to all others. Each channel includes an accumulator; allows for constant pressure pumping of all active channels simultaneously, while time-multiplexing pump-driven inflow.

In various exemplary embodiments, the manifold has reaction chambers built in for general purpose mixing and filtering operations. Each chamber has a macro-sized channel through which the manifold delivers a sample extracted from feces (filling the reaction chamber), and the chamber has a micro-sized channel. Pumps located internal or external to the manifold drive fluid into the reaction chamber, and into the micro-sized channel. A valve at the output of the macro-channel, and possibly at the output of the micro-channel, controls fluid direction as it exits the reaction chamber.

Microfluidic applications require support infrastructure for sample preparation, sample delivery, consumable storage, consumable delivery or replenishment, and waste extraction. In various exemplary embodiments, the manifold includes integrated support for differential pressure applications, pneumatic operations, sample and additive reservoirs, sample accumulators, external pumps, pneumatic pressure sources, active pump pressure (e.g., peristaltic, check-valve actuators, electro-osmotic, electrophoretic), acoustic or vibrational energy, and light-interaction (e.g., spectrometer, laser, UV, magnification). The acoustic energy source may be a high frequency (54 MHz) bulk acoustic wave (BAW) actuator.

In various exemplary embodiments, the manifold interface has a matrix of ports, possibly laid out in a regular grid. These ports may be activated or closed via an external support manifold. Routing is fully programmable.

In various exemplary embodiments, the manifold directs one or more fluids to the analytical test device 210 or MFC analytical test device 400 to cleanse the devices. These may include cleaning solutions, disinfectants, and flushing fluids. In various exemplary embodiments, the manifold directs hot water or steam to clean sample, reagents, etc. from the devices. In various exemplary embodiments, the toilet systems using oxygenated water, ozonated water, electrolyzed water, which may be generated on an as-needed basis by the toilet system (this may be internal or external to the toilet).

In various exemplary embodiments, waste from the MFCs is managed based on its characteristics and associated legal requirements. Waste that can be safely disposed is discharged into the sewer line. Waste that can be rendered chemically inert (e.g., heat treatment, vaporization, neutralization) is processed and discharged. Waste that cannot be discharged or treated in the toilet system is stored, and sequestered if necessary, for removal and appropriate handling.

In various exemplary embodiments, the manifold creates sequestered zones for each of these waste categories and ensures that all products are properly handled. In various exemplary embodiments, the manifold directs flushing water and/or cleansing fluids to clean the manifold and MFC. In some embodiments, high-pressure fluids are used for cleaning. In such an embodiment, the high-pressure fluids are not used in the MFC. In some embodiments, the MFC is removed from the backplate interface and all ports are part of the high-pressure cleansing and/or rinse.

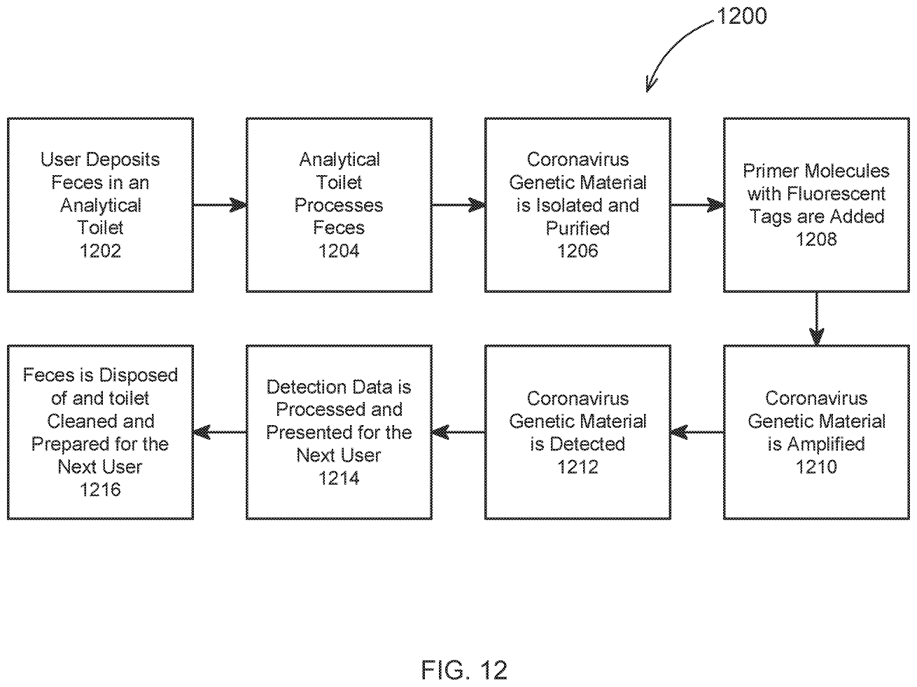

FIG. 12 illustrates the various steps of a method 1200 to detect a coronavirus in an analytical toilet, according to an embodiment of the disclosure. Detection method 1200 depicts a series of steps resulting in a positive result for detection of a coronavirus. The method comprises:
1) User Deposits Feces in an Analytical Toilet 1202;
2) Analytical Toilet Processes Feces 1204;
3) Coronavirus Genetic Material is Isolated and Purified 1206;
4) Primer Molecules with Fluorescent Tags are Added 1208;
5) Coronavirus Genetic Material is Amplified 1210;
6) Coronavirus Genetic Material is Detected 1212;
7) Detection Data is Processed and Presented to the User 1214; and
8) Feces is Disposed of and Toilet Cleaned and Prepared for the Next User 1216.

Following use of the coronavirus detection system, the toilet may prepare the system for future analysis by removing from the test area waste products and other things that might contaminate the next analysis. This could include flushing the detection system, adding a buffer or stabilizing solution, or adding a gas to remove all liquid from the system. There are various options to clean, sanitize, and/or prepare the various components of the detection system between uses of the toilet. In one preferred embodiment, hot water is run through the fluidic circuits. In another preferred embodiment, oxygenated water is run through the fluidic circuits. In yet another preferred embodiment, a gas is run through the fluidic circuits to remove any liquid from being in contact with the detection system. Alternatively, cleaning and/or preservation agents are run through the fluid circuits.

Additionally, temperature can be critical to the preparation, testing, or post processing of the detection system, the fluidic circuits, or the sample. As such, temperature controls may be included to accommodate those need. The controls could be built into the toilet, built into a fluidic circuit, or a result of adding a reagent to the sample. In one preferred embodiment, a resistive wire acts as a heat source to warm the sample and/or the detection system. PCR uses multiple thermal cycles in order to denature and cleave DNA and for amplification.

In various exemplary embodiments, the analytical toilet includes additional health and wellness sensors that may be located in a variety of location. In some embodiments, the seat may contain health and wellness sensors to measure pulse, blood pressure, blood oxygenation, electrocardiography, body temperature, body weight, excreta content, excreta weight, excreta volume, excreta temperature, excreta density, excreta flow rate, and other health and wellness indicators. In a preferred embodiment, the seat is attached to the toilet via a powered quick disconnect system that allows the seat to be interchangeable. This facilitates installing custom seats to include user-specific tests based on known health conditions. It also facilitates installing upgraded seats as sensor technology improves.

In various exemplary embodiments, the lid may contain health and wellness sensors that interact with the user's back or that analyze gases in the bowl after the lid is closed.

In various exemplary embodiments, the analytical toilet includes software and hardware controls that are pre-set so that any manufacturer can configure their devices (i.e., analytical test devices) to work in the system. In a preferred embodiment, the system includes a software stack that allows for data channels to transfer data from the sensors in the medical toilet to cloud data systems. The software and hardware controls and/or software stack may be stored in the analytical toilet or remotely. This would allow scientists to place sensors, reagents, etc. in the system to obtain data for their research. It also allows user data to be individually processed, analyzed, and delivered to the user, or their health care provider, digitally (e.g., on a phone, tablet, or computer application). The seat may also contain sensors to measure fluid levels in the toilet. This could include proximity sensors. Alternatively, tubes in fluid communication with the bowl water could be used to determine changes to bowl fluids (e.g., volume, temperature, rate of changes, etc.).

The toilet disclosed herein has many possible uses, including private and public use. Whether for use by one individual, a small group of known users, or general public use, the toilet can detect, monitor, and create one-time and/or trend data for a variety of analytes, such as biomarkers. This data can be used to prompt a user to seek additional medical, health, or wellness advice or treatment; track or monitor a user or population's known condition; and provide early detection or anticipation of a disease or another condition of which a user or population may wish to be aware.

While the present disclosure often notes the coronavirus detection system and other components supporting feces analysis are located within the toilet, it is possible that some or all of the components are located outside of the toilet. For example, the sample preparation, detection, and processing equipment may be a separate unit adjacent to the toilet which cooperates with the toilet to automatically or semi-automatically receive excreta, prepare a sample of feces for analysis, test the sample, discard the sample, and prevent cross contamination by cleaning and/or sterilizing portions of the toilet and external equipment that do any portion of the described process.

### EXAMPLES

The following example is provided as part of the disclosure as an embodiment of the present invention. As such, none of the information provided below is to be taken as limiting the scope of the invention.

### Example 1. Detecting the SARS-CoV-2 Coronavirus

Example 1 is illustrative of a preferred method of detecting the SARS-CoV-2 coronavirus. The method comprises:
1) A user deposits a sample of excreta into an analytical toilet. A 1 cm³ sample of feces is removed and the feces sample is lysed with a pulse from a 54 MHz acoustic energy source. A microfluidic system within the analytical toilet directs, rinses the lysed feces with 5 mL of HEPES buffer solution (>99.5%, 4-(2-Hydroxyethyl)piperazine-1-ethanesulfonic acid, N-(2-Hydroxyethyl)piperazine-N′-(2-ethanesulfonic acid), Millipore Sigma, St. Louis, Mo., USA), filters and transports a 1 μL sample extracted from the feces to a detection system.
2) Nucleic acid extracts of coronavirus RNA were qualified prior to construction using real-time reverse transcription polymerase chain reaction (RT-PCR) for coronavirus RNA. Oligonucleotide primers and probes were purchased from Integrated DNA Technologies, Inc. (Coralville, Iowa, USA). The core amplification reagent is TaqPath™ 1-Step RT-qPCR Master Mix (4×) (ThermoFisher, Waltham, Mass., USA). The real time RT PCR assays used is nCoVPC RNA. All reactions were prepared as 20 μL volumes to which 5 μL of nucleic acid extracts were added to make a final reaction volume of 25 μL. Amplification and detection in real time was carried out using an Mx3005P qPCR system (Agilent Technologies, Santa Clara, Calif., USA) using the FAM detector channel during the 60° C. annealing stage. Each reaction used 5 μL of sample in a final reaction volume of 25 μL. The amplification protocol for reverse transcription used an incubation step of 45° C. for 10 minutes followed by a step of 95° C. for 10 min. PCR amplification then entailed 45 cycles of 30 seconds at 90° C. and 1 minute at 60° C. Fluorescence was measured in real time using the FAM channel during the 60° C. stage.
3) The fluorimeter in the detection system detects a distinct change in fluorescence due to a hybridization event between a primer molecule with a fluorescent tag and amplified coronavirus RNA.
4) The detection system relays the computer-readable data to a processor.
5) The processor processes the data and relays the information to the user or a medical professional at an interface.
6) The user or medical professional takes appropriate action in response to the data.
7) The analytical toilet flushes and cleans the detection system and bowl in preparation for the next user.

All patents, published patent applications, and other publications referred to herein are incorporated herein by reference. The invention has been described with reference to various specific and preferred embodiments and techniques. Nevertheless, it is understood that many variations and modifications may be made while remaining within the spirit and scope of the invention.

What is claimed is:

1. An analytical toilet comprising:
a bowl adapted to receive feces;
a device to extract a sample from the feces;
a detection system to receive the sample and detect a virus; and
a passage for transferring the sample to the detection system;
wherein, when the sample is brought into contact with the detection system, the detection system indicates the presence of a virus by a distinct signal.

2. The analytical toilet of claim 1, wherein the distinct signal is an electric signal, colorimetric signal, or a spectroscopic signal.

3. The analytical toilet of claim 1, wherein a property of the distinct signal is indicative of the concentration of a virus in the sample.

4. The analytical toilet of claim 1 wherein the virus is selected from the group consisting of alphacoronavirus, betacoronavirus, gammacoronavirus, deltacoronavirus, SARS-CoV, SARS-CoV-2, and MERS-CoV.

5. The analytical toilet of claim 1, wherein the detection system comprises an amplification device to amplify virus RNA.

6. The analytical toilet of claim 1, wherein the device is a quantitative PCR instrument.

7. The analytical toilet of claim 6, wherein the detection system comprises microfluidic channels to transport the sample to the quantitative PCR instrument.

8. The analytical toilet of claim 1, wherein the detection system is capable of detecting the virus using a nucleic acid test or nucleic acid amplification test.

9. The analytical toilet of claim 1, wherein the detection system comprises a fluorimeter or primer molecules.

10. The analytical toilet of claim 1, wherein the detection system comprises primer molecules.

11. The analytical toilet of claim 10, wherein the primer molecules comprise oligonucleotides, a quencher, or a fluorescent tag.

12. The analytical toilet of claim 10, wherein the primer molecules are a fluorescent tag selected from the group consisting of 6-FAM™, JOE™, TET™, Cal Fluor Gold 540, HEX™, Cal Fluor Orange 560, TAMRA™, Cyanine 3, Quasar™ 570, Cal Fluor Red 590, ROX™, Texas Red™, Cyanine 5, Quasar™ 670, Cyanine 5.5, NED, Cal Fluor Red 610 or 635, VIC, Bioresearch Blue™, Pulsar™ 650, and Quasar™ 705.

13. The analytical toilet of claim 1, wherein the detection system uses a nucleic acid test or a nucleic acid amplification test to detect a virus.

14. The analytical toilet of claim 1, wherein the detection system can detect two or more different viruses at the same time.

15. The analytical toilet of claim 1, wherein a component of a virus is measured using a miniaturized mass spectrometer.

16. The analytical toilet of claim 1, wherein data from the detection system is used to generate individual trend data over time of the number of virus particles in the feces of a particular user.

17. The analytical toilet of claim 16, wherein the individual trend data is reported to the user to enhance health and wellness.

18. The analytical toilet analytical toilet of claim 16, wherein the individual trend data from multiple users is combined to generate population trend data.

19. The analytical toilet of claim 1, further comprising a filter to remove solid material from a sample to be transferred to a detection system.

20. A method to analyze feces comprising:
receiving feces from a user in a bowl of a toilet;
extracting a sample from the feces;
lysing material within the feces to release nucleic acids;
rinsing the lysed material with a buffer to form a rinse solution;
transferring the rinse solution through a passage to one or more detection systems comprising a quantitative PCR instrument; and
wherein, when the quantitative PCR instrument indicates the presence of a targeted genetic material within the sample.
